(19) United States
(12) Patent Application Publication
(10) Pub. No.: US 2024/0244781 A1
(43) Pub. Date: Jul. 18, 2024

(54) HEADER LASER ETCHING FEEDTHROUGH FOR CERAMIC BONDING

(71) Applicant: Cardiac Pacemakers, Inc., St. Paul, MN (US)

(72) Inventors: James Michael English, Cahir (IE); Moira B. Sweeney, St. Paul, MN (US); Benjamin J. Haasl, Forest Lake, MN (US); Dalton Michael Gorman, Andover, MN (US)

(21) Appl. No.: 18/414,209

(22) Filed: Jan. 16, 2024

Related U.S. Application Data
(60) Provisional application No. 63/439,219, filed on Jan. 16, 2023.

(51) Int. Cl. H05K 5/06; A61N 1/375; H05K 5/02; H05K 5/04

(52) U.S. Cl. CPC H05K 5/06; A61N 1/3754; H05K 5/0247; H05K 5/04

(57) ABSTRACT

A medical device comprises a metal case having a hermetic seal; one or more Input/Output (I/O) conductors, wherein the I/O conductors pass through the hermetic seal such that a first end of the I/O conductors reside on a non-hermetic side of the hermetic seal and a second end of the I/O conductors reside on a hermetic side of the hermetic seal within the metal case; a ceramic substrate, wherein the one or more I/O conductors pass through the ceramic substrate and the ceramic substrate has a non-hermetic surface on a non-hermetic side of the hermetic seal; and wherein the non-hermetic surface of the ceramic substrate is a laser etched surface.

### CLAIM OF PRIORITY

This application claims the benefit of U.S. Provisional Application No. 63/439,219 filed on Jan. 16, 2023, which is hereby incorporated by reference in its entirety.

### TECHNICAL FIELD

This patent application relates to implantable medical devices and, in particular, to hermetic seal feedthroughs.

### BACKGROUND

Implantable medical devices generally include a hermetically sealed metal case or metal housing. For implantable cardiac rhythm management devices, electrical signals sensed in the body and electrical signals delivered to the body need to pass through the hermetic seal. These electrical signals must pass through the hermetic seal of the device and yet be insulated from the metal case. This is accomplished with feedthroughs. Feedthroughs are comprised of an electrical conductor, usually a pin, passing through insulating material and providing connection from circuitry internal to the case to a point external to the case while maintaining the hermetic seal. It can be challenging to design hermetically protected circuit assemblies to operate in a body tissue environment.

### SUMMARY

Systems and methods are disclosed to feedthrough assemblies for medical devices. Example 1 includes subject matter (such as an implantable medical device) including a metal case having a hermetic seal; one or more Input/Output (I/O) conductors, wherein the I/O conductors pass through the hermetic seal such that a first end of the I/O conductors reside on a non-hermetic side of the hermetic seal and a second end of the I/O conductors reside on a hermetic side of the hermetic seal within the metal case; and a ceramic substrate. The one or more I/O conductors pass through the ceramic substrate and the ceramic substrate has a non-hermetic surface on a non-hermetic side of the hermetic seal. The non-hermetic surface of the ceramic substrate is a laser etched surface.

In Example 2, the subject matter of Example 1 optionally includes a laser etched surface of the ceramic substrate that is laser etched using a femtosecond laser.

In Example 3, the subject matter of one or both of Examples 1 and 2 optionally includes the one or more I/O conductors and the laser etched surface of the ceramic substrate covered with an epoxy.

In Example 4, the subject matter of one or any combination of Examples 1-3 optionally includes at least one of the one or more I/O conductors being electrically connected to an indifferent electrode by a wire, and the ceramic substrate, the at least one I/O conductor, and the indifferent electrode covered by the epoxy.

In Example 5, the subject matter of one or both of Examples 1 and 2 optionally includes a mold layer encapsulating the one or more I/O conductors and the laser etched surface of the ceramic substrate.

In Example 6, the subject matter of one or any combination of Examples 1-5 optionally includes a metal case including a flange, and the ceramic substrate is coupled to the flange using a gold braze on an edge of the ceramic substrate.

In Example 7, the subject matter of Example 6 optionally includes at least one of the one or more I/O conductors is electrically connected to an indifferent electrode by a wire, and an indifferent electrode arranged opposite a first surface of the metal case, and the ceramic substrate is disposed in an indentation on a second surface of the metal case.

In Example 8, the subject matter of Example 7 optionally includes a lead receptacle configured to receive an electrical lead; wherein the indifferent electrode is included in the lead receptacle.

In Example 9, the subject matter of one or any combination of Examples 1-8 optionally includes one or more I/O conductors that are metal pins, and the metal pins pass through the ceramic substrate and are coupled to the ceramic substrate using a gold braze.

In Example 10, the subject matter of Example 9 optionally includes metal pins that include at least one of platinum, iridium, or niobium.

In Example 11, the subject matter of one or any combination of Examples 1-10 optionally includes one or more a lead receptacles. Each lead receptacle is configured to receive an electrical lead, and the I/O conductors are electrically connected to electrodes of the one or more lead receptacles.

Example 12 includes subject matter (such as a method of forming a feedthrough for an implantable medical device) or can optionally be combined with one or any combination of Examples 1-11 to include such subject matter, including forming a ceramic substrate having multiple openings; disposing multiple I/O conductors in the multiple openings of the ceramic substrate; coupling the I/O conductors to the ceramic substrate by brazing; and laser etching a surface of the ceramic substrate.

In Example 13, the subject matter of Example 12 optionally includes laser etching the surface of the ceramic substrate using a femtosecond laser.

In Example 14, the subject matter of one or both of Examples 12 and 13 optionally includes brazing the ceramic substrate to a metal case of the implantable medical device to form a hermetic seal for the implantable medical device.

In Example 15, the subject matter of one or any combination of Examples 12-14 optionally includes covering the I/O conductors and laser-etched ceramic substrate with an epoxy.

In Example 16, the subject matter of Example 15 optionally includes disposing an indifferent electrode opposite a first surface of the metal case of the implantable medical device; disposing the ceramic substrate in an indentation of a second surface of the metal case; and connecting the indifferent electrode to at least one of the one or more I/O conductors using a wire.

In Example 17, the subject matter of Example 16 optionally includes covering the ceramic substrate, the at least one of the one or more I/O conductors, and the indifferent electrode with an epoxy.

Example 18 includes subject matter (such as an apparatus) or can optionally be combined with one or any combination of Examples 1-17 to include such subject matter, including a ceramic substrate having multiple openings and multiple I/O conductors disposed in the multiple openings of the ceramic substrate. The I/O conductors are brazed to the ceramic substrate. A first surface of ceramic substrate is laser etched and a second surface of the ceramic substrate is smooth.

In Example 19, the subject matter of Example 18 optionally includes the first surface of the ceramic substrate being laser etched using a femtosecond laser.

In Example 20, the subject matter of one or both of Examples 18 and 19 optionally includes a metal case that includes a metal flange, and a ceramic substrate brazed to the metal flange such that the first surface of the ceramic substrate is external to the metal case and the second surface of the ceramic substrate is internal to the metal case.

The non-limiting Examples can be combined in any permutation or combination. This summary is intended to provide an overview of the subject matter of the present application. It is not intended to provide an exclusive or exhaustive explanation of the invention. The detailed description is included to provide further information about the subject matter of the present patent application.

### BRIEF DESCRIPTION OF THE DRAWINGS

FIG. 1 is an illustration of portions of a system that uses a medical device.

FIG. 2 is a view of another example of a medical device.

FIG. 3 is a view of an example of a feedthrough assembly.

FIG. 4 is a flow diagram of an example of a method of manufacture for a feedthrough assembly.

### DETAILED DESCRIPTION

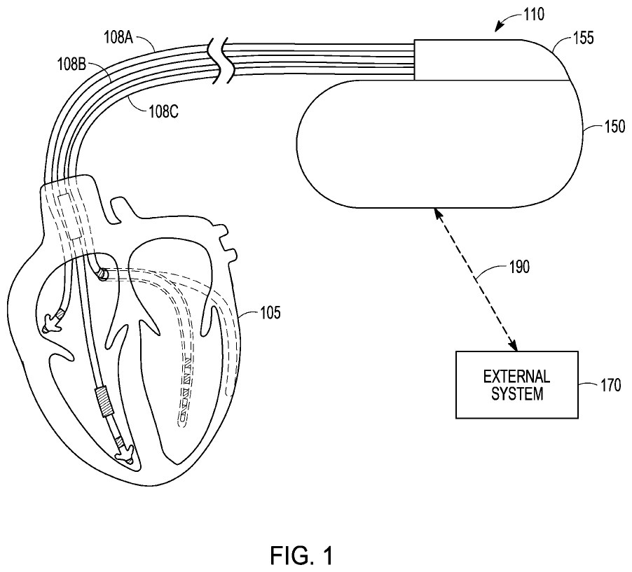

This document relates to a connector assembly for an ambulatory medical device. FIG. 1 is an illustration of portions of a system that uses an implantable medical device (IMD). Some examples of the IMD 110 include a pacemaker, a defibrillator, a cardiac resynchronization therapy (CRT) device, a combination of such devices, or a diagnostic-only device. The system also typically includes an IMD programmer or other external device 170 that communicates wireless signals 190 with the IMD 110, such as by using radio frequency (RF) or other telemetry signals.

The IMD 110 can be coupled by one or more conductive leads 108A-C to heart 105. The cardiac leads 108A-C in the example of FIG. 1 include a proximal end that is coupled to IMD 110 and a distal end, coupled by electrical contacts or “electrodes” to one or more portions of a heart 105. The electrodes typically deliver cardioversion, defibrillation, pacing, or resynchronization therapy, or combinations thereof to at least one chamber of the heart 105. The electrodes may be electrically coupled to sense amplifiers to sense electrical cardiac signals. Sensed electrical cardiac signals can be sampled to create an electrogram. An electrogram can be analyzed by the IMD and/or can be stored in the IMD and later communicated to an external device where the sampled signals can be displayed for analysis.

The cardiac leads 108A-C include right atrial (RA) lead 108A, right ventricle (RV) lead 108B, and a third cardiac lead 108C for placement in a coronary vein lying epicardially on the left ventricle (LV) via the coronary vein.

The IMD 110 includes a hermetically-sealed metal housing or metal case 150 that houses electronic circuits and a header connector 155. Conductive leads (e.g., cardiac leads 108A-C) are connected to the IMD through the header connector 155. The header connector 155 includes lead receptacles for the cardiac leads 108A-C. The lead receptacles can be encapsulated in a mold material (e.g., an epoxy).

For implantable cardiac rhythm management devices, electrical signals sensed in the body and electrical signals delivered to the body need to pass through the hermetic seal. This is accomplished with feedthroughs. Feedthroughs are comprised of an electrical conductor, usually a pin, passing through insulating material and providing connection from circuitry internal to the case to a point external to the case (e.g., an electrode of a lead receptacle) while maintaining the hermetic seal. The header connector 155 provides the electrical connection between conductive leads and the feedthroughs.

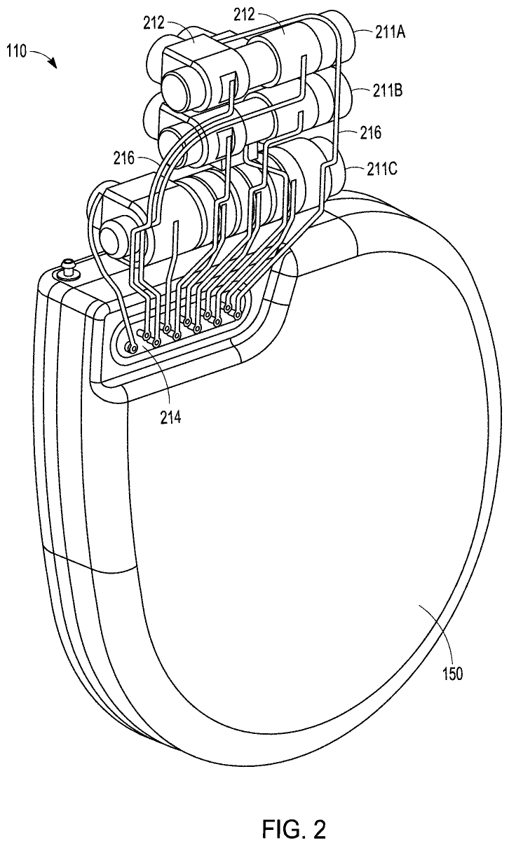

FIG. 2 is a view of another example of a medical device 110. The view shows the metal case 150 of the medical device and shows the header connector without the mold material to expose the lead receptacles. The header connector includes three lead receptacles 211A-C into which conductive leads can be inserted so that electrodes of the conductive leads contact electrodes 212 of the lead receptacles 211A-C. FIG. 2 also shows a feedthrough connector assembly 214 of the medical device 110. The feedthroughs are connected to the lead receptacles 211A-C by conductive wires 216. The lead receptacles 211A-C are arranged above a top surface of the metal case 150, and the feedthrough connector assembly 214 is arranged in an indentation of the side surface of the metal case 150. The indentation allows the feedthroughs of the feedthrough assembly 214 to extend out the side of the metal case 150 without increasing the width of the medical device 110.

The medical device 110 is implantable in body tissue. Body tissue is a high-moisture environment that can cause failures in medical devices 110. For example, moisture can get inside the lead receptacles 211A-C. If moisture gets to the outside of the lead receptacles under the mold material, it can get to the feedthrough assembly 214 along the wires 216 even though the outside of the lead receptacles is encapsulated. For example, moisture could travel from an indifferent electrode of a lead receptacle along a wire 216 to the feedthrough connector assembly 214 even though the lead receptacles 211A-C, wires 216, and the feedthrough connector assembly 214 are all covered with an epoxy. The medical device 110 may be capable of delivering defibrillation energy to a conductive lead. If moisture is present at the feedthrough or feedthroughs providing the defibrillation energy, the moisture may vaporize, and the vapor may damage one or more feedthrough connections.

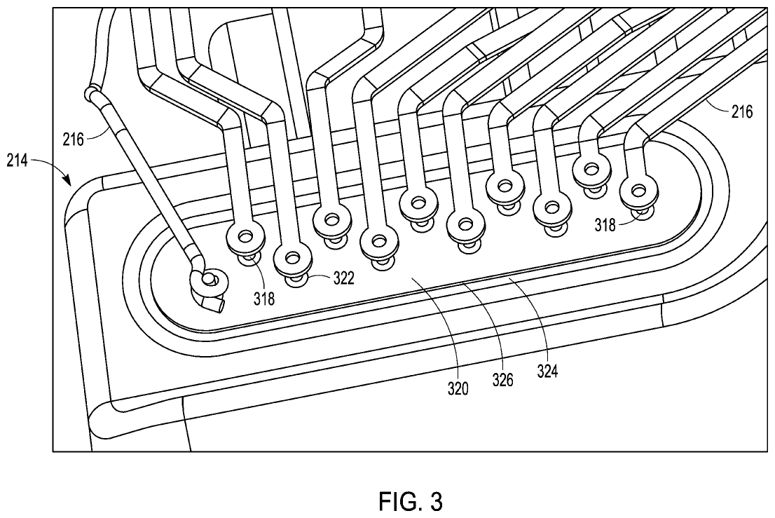

FIG. 3 is another view of the feedthrough assembly 214 of FIG. 2. The feedthroughs include multiple Input/Output (I/O) conductors 318 that pass through the external part of the header connector to inside the hermetically sealed environment within the metal case 150 so that one end of the I/O conductors 318 is on a non-hermetic side of the hermetic seal and the other of the I/O conductors 318 is on a hermetic side of the hermetic seal.

The feedthrough assembly 214 includes a ceramic substrate 320. The I/O conductors 318 pass through openings in the ceramic substrate 320 and are coupled to the ceramic substrate 320. In some examples, the I/O conductors 318 include metal pins that are coupled to the ceramic substrate 320 by brazing. In certain examples, the I/O conductors 318 include one or more of platinum, iridium, or niobium and are coupled to the ceramic substrate with a gold braze 322. The metal case 150 may include a flange 324. The ceramic substrate is coupled to the flange using a braze 326 around the edge of the ceramic substrate 320. In certain examples, the braze 326 around the edge of the ceramic substrate 320 can include gold.

The ceramic substrate 320 has an outside non-hermetic surface and an inside hermetic surface. A ceramic substrate is typically formed by injection-molding and then grinding to smooth the surfaces. For the ceramic substrate 320, the outside non-hermetic surface of the ceramic substrate 320 is laser etched to roughen and texture the surface. The inside hermetic surface may be smooth. In some examples, the non-hermetic surface of the ceramic substrate 320 is laser etched using a femtosecond laser. A femtosecond laser can apply pulsed laser energy having a pulse duration in the femtosecond range. The laser etching may use lower energy laser pulses delivered with very high frequency. Using a femtosecond laser has the advantage that the laser doesn't interact with the ceramic. The result is that the ceramic substrate 320 is etched cleanly, meaning that the non-hermetic surface of the ceramic substrate 320 is free of ceramic particles or organic material after the laser etching. The laser etched surface provides for better adhesion of the encapsulant (e.g., epoxy) to the ceramic substrate 320 and increases capillary action or wicking of any moisture reaching the ceramic substrate 320. This reduces mechanical failures of the feedthroughs from moisture.

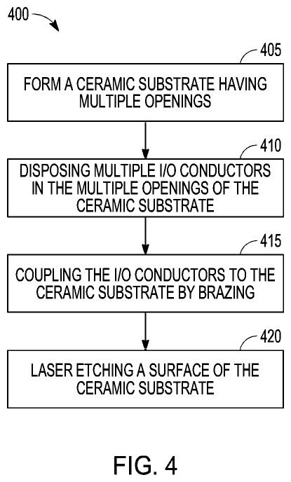

FIG. 4 is a flow diagram of an example of a method 400 of manufacture for a feedthrough assembly, such as the feedthrough assembly 214 of FIG. 3. At block 405, a ceramic substrate 320 having multiple openings is formed. The ceramic substrate 320 may include aluminum oxide.

At block 410, multiple I/O conductors 318 are disposed in the multiple openings of the ceramic substrate 320. At block 415, the I/O conductors are coupled to the ceramic substrate by brazing. In some examples, the I/O conductors 318 are metal pins that can include one or more of platinum, iridium, and niobium, and the metal pins are gold brazed to the ceramic substrate 320. At block 420, a surface of the ceramic substrate 320 is laser etched. The surface may be laser etched using a femtosecond laser.

The feedthrough assembly 214 with the etched ceramic substrate 320 can be included in a medical device. The feedthrough assembly 214 may be brazed to a welded metal case 150 of the medical device with the etched surface outside the metal case 150. The opposite surface of the etched ceramic substrate 320 may be smooth. The feedthrough assembly may be brazed to the metal case 150 in an indent of the metal case 150. The brazed feedthrough assembly 214 and the welded metal case 150 may form a hermetic seal for the medical device. The I/O conductors 318 and the laser-etched ceramic substrate 320 can be covered with an epoxy.

In some examples, the medical device includes one or more lead receptacles 211. The lead receptacles include electrodes to contact electrodes of conductive leads when the leads are inserted into the lead receptacles. The electrodes can be electrically connected to the I/O conductors 318 using conductive wires 216, and the electrodes, I/O conductors 318, etched ceramic substrate 320, and conductive wires 216 can be covered with epoxy.

The devices described herein provide a header connector for a medical device that is robust to body implantation.

### Additional Description

The above detailed description includes references to the accompanying drawings, which form a part of the detailed description. The drawings show, by way of illustration, specific embodiments in which the invention can be practiced. These embodiments are also referred to herein as “examples.” All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times. These computer-readable media can include, but are not limited to, hard disks, removable magnetic disks, removable optical disks (e.g., compact disks and digital video disks), magnetic cassettes, memory cards or sticks, random access memories (RAM's), read only memories (ROM's), and the like. In some examples, a carrier medium can carry code implementing the methods. The term “carrier medium” can be used to represent carrier waves on which code is transmitted.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

What is claimed is:

1. An implantable medical device, the device comprising:
a metal case having a hermetic seal;
one or more Input/Output (I/O) conductors, wherein the I/O conductors pass through the hermetic seal such that a first end of the I/O conductors reside on a non-hermetic side of the hermetic seal and a second end of the I/O conductors reside on a hermetic side of the hermetic seal within the metal case;
a ceramic substrate, wherein the one or more I/O conductors pass through the ceramic substrate and the ceramic substrate has a non-hermetic surface on a non-hermetic side of the hermetic seal; and
wherein the non-hermetic surface of the ceramic substrate is a laser etched surface.

2. The device of claim 1, wherein the laser etched surface of the ceramic substrate is laser etched using a femtosecond laser.

3. The apparatus of claim 1, wherein the one or more I/O conductors and the laser etched surface of the ceramic substrate are covered with an epoxy.

4. The device of claim 3, wherein at least one of the one or more I/O conductors is electrically connected to an indifferent electrode by a wire, and the ceramic substrate, the at least one I/O conductor, and the indifferent electrode are covered by the epoxy.

5. The device of claim 1, including a mold layer encapsulating the one or more I/O conductors and the laser etched surface of the ceramic substrate.

6. The device of claim 1,
wherein the metal case includes a flange; and
wherein the ceramic substrate is coupled to the flange using a gold braze on an edge of the ceramic substrate.

7. The device of claim 6,
wherein at least one of the one or more I/O conductors is electrically connected to an indifferent electrode by a wire; and
wherein the indifferent electrode is arranged opposite a first surface of the metal case, and the ceramic substrate is disposed in an indentation on a second surface of the metal case.

8. The device of claim 7, including a lead receptacle configured to receive an electrical lead; wherein the indifferent electrode is included in the lead receptacle.

9. The device of claim 1, wherein the one or more I/O conductors are metal pins, and the metal pins pass through the ceramic substrate and are coupled to the ceramic substrate using a gold braze.

10. The device of claim 9, wherein the metal pins include at least one of platinum, iridium, or niobium.

11. The device of claim 1, including one or more a lead receptacles each configured to receive an electrical lead, wherein the I/O conductors are electrically connected to electrodes of the one or more lead receptacles.

12. A method of forming a feedthrough for an implantable medical device, the method comprising:
forming a ceramic substrate having multiple openings;
disposing multiple I/O conductors in the multiple openings of the ceramic substrate;
coupling the I/O conductors to the ceramic substrate by brazing; and
laser etching a surface of the ceramic substrate.

13. The method of claim 12, wherein laser etching the surface of the ceramic substrate includes laser etching the surface of the ceramic substrate using a femtosecond laser.

14. The method of claim 12, including brazing the ceramic substrate to a metal case of the implantable medical device to form a hermetic seal for the implantable medical device.

15. The method of claim 12, including covering the I/O conductors and laser-etched ceramic substrate with an epoxy.

16. The method of claim 15, including:
disposing an indifferent electrode opposite a first surface of the metal case of the implantable medical device;
disposing the ceramic substrate in an indentation of a second surface of the metal case; and
connecting the indifferent electrode to at least one of the one or more I/O conductors using a wire.

17. The method of claim 16, including covering the ceramic substrate, the at least one of the one or more I/O conductors, and the indifferent electrode with an epoxy.

18. An apparatus comprising:
a ceramic substrate having multiple openings;
multiple I/O conductors disposed in the multiple openings of the ceramic substrate, wherein the I/O conductors are brazed to the ceramic substrate; and
wherein a first surface of ceramic substrate is laser etched and a second surface of the ceramic substrate is smooth.

19. The apparatus of claim 18, wherein the first surface of the ceramic substrate is laser etched using a femtosecond laser.

20. The apparatus of claim 18, including:
a metal case that includes a metal flange; and
wherein the ceramic substrate is brazed to the metal flange such that the first surface of the ceramic substrate is external to the metal case and the second surface of the ceramic substrate is internal to the metal case.
